(19) United States
(12) Patent Application Publication
(10) Pub. No.: US 2010/0227891 A1
(43) Pub. Date: Sep. 9, 2010

(54) THRESHOLD BLOOD OMEPRAZOLE CONCENTRATION IS 50 NG/ML FOR THE MAINTENANCE OF INTRAGASTRIC PH OF AT LEAST 4.0 AFTER ORAL DOSING WITH CMA-OMEPRAZOLE, AGN 201904-Z

(76) Inventors: Edward Lee, Los Angeles, CA (US); Dale K. Yu, Silverado, CA (US); John Sefton, Trabuco Canyon, CA (US); Dari D. Parizadeh, Aliso Viejo, CA (US); Diane Tang-Liu, Newport Beach, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/577,800

(22) Filed: Oct. 13, 2009

Related U.S. Application Data
(63) Continuation of application No. 11/734,691, filed on Apr. 12, 2007, now abandoned.
(60) Provisional application No. 60/805,166, filed on Jun. 19, 2006.

(51) Int. Cl. A61K 31/4439; C07D 401/12; A61P 1/04

(52) U.S. Cl. 514/338; 546/273.7

(57) ABSTRACT

Dosage forms of sodium {4-[5-Methoxy-2-(4-methoxy-3,5-dimethyl-pyridin-2-ylmethanesulfinyl)-benzoimidazole-1-sulfonyl]-phenoxy}-acetate, and methods of use of the dosage forms are disclosed herein.

### CROSS-REFERENCE TO RELATED APPLICATION

This application is based on, and claims priority under 35 U.S.C. § 120 to U.S. Provisional Patent Application No. 60/805,166, filed on Jun. 19, 2006, and which is incorporated herein by reference.

Purpose

To determine the threshold blood omeprazole concentration for the maintenance of intragastric ph of at least 4.0 after oral dosing with AGN 201904-Z, an acid-stable, chemically metered absorption derivative of omeprazole (CMA-omeprazole).

Methods

Forty healthy male subjects received 240, 480, and 640 mg AGN 201904-Z or 40 mg esomeprazole orally once-daily for 5 days in a open-label, randomized, 4-way cross-over study. These doses were estimated to deliver molar equivalent omeprazole doses of 53%, 107%, and 143% of the esomeprazole dose, respectively. Pharmacokinetic (PK) blood samples were collected at 8 timepoints following the 1^{st} and 5^{th} dose of each treatment. Pharmacodynamics (PD) were evaluated via 24-hr intragastric ph monitoring before treatment and after the 1^{st} and 5^{th} doses of each treatment. Empirical PK/PD models were constructed for AGN 201904-Z data by regressing the % time that intragastric pH ≧4.0 during a 24-hr dosing period against the duration of time that blood omeprazole concentrations were greater than 8 different concentrations over the same 24-hr period. The possible threshold concentrations evaluated ranged from 10 to 400 ng/mL. The concentration that provided the best-fit was designated the threshold concentration. The best-fit concentration and model was chosen based on the WSSR, R², visual inspection, and AIC value.

Results

Among the possible omeprazole threshold concentrations evaluated, 50 ng/mL was the best-fit concentration value in all of the models evaluated. The final model suggests the % time intragastric pH ≧4.0 is dependent on the duration of time that blood omeprazole concentrations are maintained above 50 ng/mL. This agreed with the actual results which showed AGN 201904-Z maintained drug concentrations above 50 ng/mL longer than esomeprazole (18.0 vs. 9.2 hr), and also maintained intragastric pH>4.0 longer than esomeprazole (80.1 vs. 68.4% of 24-hr; p<0.0001).

Conclusion

The 50 ng/mL blood omeprazole concentration provided the best correlation with the percent time intragastric pH was ≧4.0 over a 24-hr dosing interval and was designated as the threshold concentration of omeprazole AGN 201904-Z, a CMA-omeprazole designed to provide sustained omeprazole exposure, was more effective than esomeprazole at maintaining 24-hour intragastric ph ≧4.0.

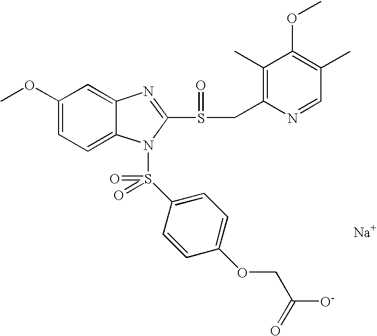

AGN 201904-Z, sodium {4-[5-Methoxy-2-(4-methoxy-3,5-dimethyl-pyridin-2-ylmethanesulfinyl)-benzoimidazole-1-sulfonyl]-phenoxy}-acetate

What is claimed is:

1. A dosage form containing from about 200 to about 700 mg of sodium {4-[5-Methoxy-2-(4-methoxy-3,5-dimethyl-pyridin-2-ylmethanesulfinyl)-benzoimidazole-1-sulfonyl]-phenoxy}-acetate.

2. The dosage form of claim 1 wherein said dosage form contains about 240 mg of sodium {4-[5-Methoxy-2-(4-methoxy-3,5-dimethyl-pyridin-2-ylmethanesulfinyl)-benzoimidazole-1-sulfonyl]-phenoxy}-acetate.

3. The dosage form of claim 1 wherein said dosage form contains about 480 mg of sodium {4-[5-Methoxy-2-(4-methoxy-3,5-dimethyl-pyridin-2-ylmethanesulfinyl)-benzoimidazole-1-sulfonyl]-phenoxy}-acetate.

4. The dosage form of claim 1 wherein said dosage form contains about 640 mg of sodium {4-[5-Methoxy-2-(4-methoxy-3,5-dimethyl-pyridin-2-ylmethanesulfinyl)-benzoimidazole-1-sulfonyl]-phenoxy}-acetate.

5. A method of maintaining stomach pH of a person at or above 4, comprising maintaining the concentration of omeprazole in the blood of a person at or above 50 ng/mL for at least 17 hours during a 24 hour period.

6. The method of claim 5 further comprising orally administering a dosage form of any one of claims 1 to 4 to said person once a day.

7. The method of claim 5 further comprising administering from about 200 to about 700 mg of sodium {4-[5-Methoxy-2-(4-methoxy-3,5-dimethyl-pyridin-2-ylmethanesulfinyl)-benzoimidazole-1-sulfonyl]-phenoxy}-acetate orally to the person once a day.
